(19) United States
(12) Patent Application Publication
(10) Pub. No.: US 2022/0089883 A1
(43) Pub. Date: Mar. 24, 2022

(54) ANTIMICROBIAL ELASTOMERIC ARTICLE

(71) Applicant: Top Glove International Sdn. Bhd., Klang (MY)

(72) Inventors: Chong Ban Wong, Klang (MY); Nuriah Mohamad, Klang (MY); Devi Shantini P.Chandrasakaran, Klang (MY); Anuja Selvadurai, Klang (MY); Rovethra Subramaniam, Klang (MY)

(21) Appl. No.: 17/483,140

(22) Filed: Sep. 23, 2021

(30) Foreign Application Priority Data
Sep. 24, 2020 (MY) PI2020004969

(51) Int. Cl. C09D 5/14; C09D 7/63; C09D 7/61; A01N 65/26; A01N 65/36; A41D 19/00

(52) U.S. Cl. CPC C09D 5/14; C09D 7/63; A41D 19/0055; A01N 65/26; A01N 65/36; C09D 7/61

(57) ABSTRACT

Described herein is antimicrobial coagulant solution. In certain embodiments, the antimicrobial coagulant solution is a mixture of an antimicrobial additive and a coagulant solution. In certain embodiments, the antimicrobial additive is a neem extract, a lemon extract, or combination thereof. The coagulant solution includes an antitack agent, a coagulating agent, a wetting agent, a pH adjuster and a solvent.

### CROSS-REFERENCE TO RELATED APPLICATIONS

The present application claims priority under 35 U.S.C. § 119(a) to Malaysia Patent Application No. P12020004969, filed on Sep. 24, 2020, which is incorporated herein by reference in its entirety.

### BACKGROUND

A primary concern in the healthcare environment is protecting the patient from endogenous, exogenous or nosocomial infections. Elastomeric article, in particular glove has become an element of personal protective equipment and essential part of clinical practice for healthcare workers. It actually safeguards both glove wearer and patient from infection.

Antimicrobial gloves are useful for reducing nosocomial infection by bacteria, fungi and viruses. An unresolved problem for developing antimicrobial gloves is the difficulty of maintaining the antimicrobial efficacy during and after production process.

Therefore, there is a need for developing antimicrobial gloves that are able to maintain the antimicrobial efficacy during and after production process. The instant specification addresses this need.

### SUMMARY

The present invention relates in one aspect to antimicrobial elastomeric articles wherein the antimicrobial elastomeric articles having antimicrobial coating. The antimicrobial coating is prepared from antimicrobial coagulant solution which comprises antimicrobial additive derived from plant.

The instant specification is directed to an antimicrobial coagulant solution. In some embodiments, the antimicrobial coagulant solution is a mixture of an antimicrobial additive and a coagulant solution. In some embodiments, the antimicrobial additive is selected from the group consisting of neem extract or lemon extract. In some embodiments, the coagulant solution comprises an antitack agent, a coagulating agent, a wetting agent, a pH adjuster and a solvent. In some embodiments, the instant specification is further directed to an antimicrobial elastomeric article having antimicrobial coating that is prepared from the antimicrobial coagulant solution. In some embodiments, the antimicrobial elastomeric article is an antimicrobial glove.

Furthermore, the instant specification is directed to the embodiments below:

Embodiment 1 provides an antimicrobial coagulant solution which is a mixture of an antimicrobial additive and a coagulant solution, wherein the antimicrobial additive is selected from the group consisting of neem extract or lemon extract and wherein the coagulant solution comprises an antitack agent, a coagulating agent, a wetting agent, a pH adjuster and a solvent.

Embodiment 2 provides the antimicrobial coagulant solution of embodiment 1, wherein the neem extract solution is used in an amount ranging between 0.40% to 1.00% by w/v in the antimicrobial coagulant solution.

Embodiment 3 provides the antimicrobial coagulant solution of embodiment 1, wherein the lemon extract solution is used in an amount ranging between 1.00% to 1.80% by w/v in the antimicrobial coagulant solution.

Embodiment 4 provides the antimicrobial coagulant solution of embodiment 1, wherein the neem extract solution comprises of neem extract powder and solvent.

Embodiment 5 provides the antimicrobial coagulant solution of embodiment 4, wherein the neem extract powder is used in an amount ranging between 20.0% to 50.0% by w/v in the neem extract solution.

Embodiment 6 provides the antimicrobial coagulant solution of embodiment 4, wherein the solvent is used in an amount ranging between 50.0% to 80.0% by v/v in the neem extract solution.

Embodiment 7 provides the antimicrobial coagulant solution of embodiment 1, wherein the lemon extract solution comprises of lemon extract powder and solvent.

Embodiment 8 provides the antimicrobial coagulant solution of embodiment 7, wherein the lemon extract powder is used in an amount ranging between 1.0% to 20.0% by w/v in the lemon extract solution.

Embodiment 9, the antimicrobial coagulant solution of embodiment 7, wherein the solvent is used in an amount ranging between 80.0% to 99.0% by v/v in the lemon extract solution.

Embodiment 10 provides the antimicrobial coagulant solution of embodiment 1, wherein the antitack agent is any one selected from the group consisting of potassium stearate, calcium stearate and any combinations thereof.

Embodiment 11 provides the antimicrobial coagulant solution of embodiment 1, wherein the antitack agent is used in an amount ranging between 0.50% to 2.00% by weight of the coagulant solution.

Embodiment 12 provides the antimicrobial coagulant solution of embodiment 1, wherein the coagulating agent is any one selected from the group consisting of calcium nitrate, calcium chloride and any combinations thereof.

Embodiment 13 provides the antimicrobial coagulant solution of embodiment 1, wherein the coagulating agent is used in an amount ranging between 7.50% to 8.50% by weight of the coagulant solution.

Embodiment 14 provides the antimicrobial coagulant solution of embodiment 1, wherein the wetting agent is any one selected from the group consisting of isotridecanol ethoxylates, dihexylsulfosuccinate and any combinations thereof.

Embodiment 15 provides the antimicrobial coagulant solution of embodiment 1, wherein the wetting agent is used in an amount ranging between 0.01% to 0.10% by weight of the coagulant solution.

Embodiment 16 provides the antimicrobial coagulant solution of embodiment 1, wherein the pH adjuster is any one selected from the group consisting of ammonia, potassium hydroxide, sodium hydroxide, magnesium hydroxide, ammonium bicarbonate, ammonium chloride and any combinations thereof.

Embodiment 17 provides the antimicrobial coagulant solution of embodiment 1, wherein the pH adjuster is used in an amount ranging between 0.001% to 0.010% by weight of the coagulant solution.

Embodiment 18 provides the antimicrobial coagulant solution of embodiment 1, wherein the solvent is any one selected from the group consisting of tap water, distilled water, deionized water and any combinations thereof.

Embodiment 19 provides the antimicrobial coagulant solution of embodiment 1, wherein the solvent is used in an amount to achieve 100% by weight of the coagulant solution.

Embodiment 20 provides an antimicrobial elastomeric article includes at least one layer of coating, wherein the coating is antimicrobial coating and wherein the antimicrobial coating is prepared from the antimicrobial coagulant solutions of embodiment 1.

Embodiment 21 provides the antimicrobial elastomeric article of embodiment 20, which is an antimicrobial glove.

### BRIEF DESCRIPTION OF THE ACCOMPANYING DRAWINGS

The instant specification will be fully understood from the detailed description given herein below and the accompanying drawings which are given by way of illustration.

It should be understood, however, that the instant specification is not limited to the precise arrangements and instrumentalities of the embodiments shown in the drawings.

FIG. 1 depicts a flow diagram of a method for producing an antimicrobial glove in accordance with some embodiments.

### DETAILED DESCRIPTION

Detailed description of embodiments is disclosed herein. It should be understood, however, that the embodiments are merely exemplary, which may be embodied in various forms. Therefore, the details disclosed herein are not to be interpreted as limiting, but merely as the basis for the claims and for teaching one skilled in the art. The numerical data or ranges used in the specification are not to be construed as limiting. The following detailed description of the preferred embodiments will now be described in accordance with the attached drawings.

It was identified that the issues of the antimicrobial gloves as discussed in the Background section are, at least partly, caused by that the antimicrobial additive coated on the glove surface is not chemically bound and easily detach from the glove surface. Furthermore, the antimicrobial additive will also degrade during the production process due to high temperature and the inability to tolerate the coagulant solution. As a result, the amount of the antimicrobial additive on the glove surface is significantly reduced during the production process and during the cause of usage, which eventually causes the antimicrobial efficacy of the glove to reduce, as well.

To overcome the above shortcomings, an approach is developed to produce antimicrobial gloves which are able to tolerate with the coagulant solution and meet the desired antimicrobial efficacy by using antimicrobial additive derived from plant.

In some embodiments, the instant specification is directed to antimicrobial elastomeric articles, wherein the antimicrobial elastomeric articles having antimicrobial coating. The antimicrobial coating is prepared from antimicrobial coagulant solution which includes antimicrobial additive derived from plant. The antimicrobial elastomeric article is an antimicrobial glove.

As used herein, the term “coating” is related to a formation of a layer of film on a glove former at multiple stages during the manufacturing process to yield a finished glove product. The finished glove product is formed from more than one layer of coating, for instance coagulant coating, latex coating and so on.

In some embodiments, the instant specification is directed to an antimicrobial coagulant solution wherein the antimicrobial coagulant solution is a mixture of an antimicrobial additive and a coagulant solution.

In some embodiments, the antimicrobial additive is derived from a plant, wherein the antimicrobial additive is a plant extract, such as, but not limited to, a neem extract or a lemon extract. As an example, the two exemplary antimicrobial coagulant solutions below will be described in detail:
(i) neem extract based coagulant solution; and
(ii) lemon extract based coagulant solution.

As used herein, the term “antimicrobial additive” signifies the antimicrobial property exhibited by plant extract which is able to kill or suppress the growth of microorganisms.

In some embodiments, the plant extract is prepared by dissolving a plant extract powder in a solvent to produce a solution with undissolved material. Thereafter, the solution with undissolved material is filtered to yield an extract solution. In some embodiments, the extract solution is a neem extract solution or a lemon extract solution. The solvent being used to obtain neem extract solution includes distilled water, tap water, deionized water or any combinations thereof. In some embodiments, distilled water is the preferred extract solution for obtaining the neem extract solution. The solvent being used to obtain lemon extract solution includes ethanol, methanol or combination thereof. In some embodiments, ethanol is the preferred extract solution for obtaining the lemon extract solution. The undissolved material is the leftover plant extract powder that has not dissolved.

Table 1 shows an exemplary formulation of the neem extract solution.

TABLE 1
Formulation of the neem extract solution

| | | Working | | Exemplary |
|---|---|---|---|---|
| Preferred | Alternative | range | Preferred | value |
| chemical | chemical | (%) | range (%) | (%) |
| Neem | Indian | 20.0 to | 26.0 to | 28.50 |
| extract | pennywort | 50.0 | 35.0 | |
| powder | extract | | | |
| (w/v) | | | | |
| Distilled | Tap water | 50.0 to | 65.0 to | 71.50 |
| water | or deionized | 80.0 | 74.0 | |
| (v/v) | water | | | |

In some embodiments, thereafter, the neem extract solution is mixed with a coagulant solution to produce the neem extract based coagulant solution. In some embodiments, the neem extract solution is used in an amount ranging from 0.40% to 1.00% by w/v in the neem extract based coagulant solution. In some embodiments, the neem extract solution is used in an amount ranging from 0.60% to 0.80% by w/v in the neem extract based coagulant solution. In some embodiments, the neem extract solution is used in an amount of 0.70% by w/v in the neem extract based coagulant solution.

Table 2 shows an exemplary formulation of the lemon extract solution.

TABLE 2
Formulation of the lemon extract solution

| | | Working | Preferred | Exemplary |
|---|---|---|---|---|
| Preferred | Alternative | range | range | value |
| chemical | chemical | (%) | (%) | (%) |
| Lemon | Orange | 1.0 to | 4.00 to | 6.00 |
| extract | extract | 20.0 | 11.0 | |
| powder | | | | |
| (w/v) | | | | |
| Ethanol | Methanol | 80.0 to | 89.0 to | 94.0 |
| (v/v) | | 99.0 | 96.0 | |

In some embodiments, thereafter, the lemon extract solution is mixed with the coagulant solution to produce the lemon extract based coagulant solution. In some embodiments, the lemon extract solution is used in an amount ranging from 1.00% to 1.80% by w/v in the lemon extract based coagulant solution. In some embodiments, the lemon extract solution is used in an amount ranging from 1.30% to 1.60% by w/v in the lemon extract based coagulant solution. In some embodiments, the lemon extract solution is used in an amount of 1.50% by w/v in the lemon extract based coagulant solution.

In some embodiments, the coagulant solution includes an antitack agent, a coagulating agent, a wetting agent, a pH adjuster, a solvent, or any combinations thereof. In some embodiments, the coagulant solution includes the antitack agent, the coagulating agent, the wetting agent, the pH adjuster, and the solvent.

In some embodiments, the coagulant solution includes the antitack agent. In some embodiments, the antitack agent includes potassium stearate, calcium stearate or combination thereof. In some embodiments, potassium stearate is preferably included as the antitack agent. In some embodiments, the antitack agent is used in an amount ranging from 0.50% to 2.00% by weight of the coagulant solution. In some embodiments, the antitack agent is used in an amount ranging from 0.80% to 1.30% by weight of the coagulant solution. In some embodiments, the antitack agent is used in an amount of 1.00% by weight of the coagulant solution.

In some embodiments, the coagulant solution includes the coagulating agent. In some embodiments, the coagulating agent includes calcium nitrate, calcium chloride or combination thereof. In some embodiments, calcium nitrate is preferably included as the coagulating agent. In some embodiments, the coagulating agent is used in an amount ranging from 7.50% to 8.50% by weight of the coagulant solution. In some embodiments, the coagulating agent is used in an amount from 7.80% to 8.20% by weight of the coagulant solution. In some embodiments, the coagulating agent is used in an amount of 8.00% by weight of the coagulant solution.

In some embodiments, the coagulant solution includes the wetting agent. In some embodiments, the wetting agent includes isotridecanol ethoxylates, dihexylsulfosuccinate, or combination thereof. In some embodiments, isotridecanol ethoxylates preferably included as the coagulating agent. In some embodiments, the wetting agent is used in an amount ranging from 0.01% to 0.10% by weight of the coagulant solution. In some embodiments, wetting agent is used in an amount ranging from 0.03% to 0.08% by weight of the coagulant solution. In some embodiments, the wetting agent is used in an amount of 0.05% by weight of the coagulant solution.

In some embodiments, the coagulant solution includes the pH adjuster. In some embodiments, the pH adjust includes ammonia, potassium hydroxide, sodium hydroxide, magnesium hydroxide, ammonium bicarbonate, ammonium chloride or any combinations thereof. In some embodiments, ammonia is preferably included as the pH adjuster. In some embodiments, the pH adjuster is used in an amount ranging from 0.001% to 0.010% by weight of the coagulant solution. In some embodiments, the pH adjuster is used in an amount ranging from 0.003% to 0.007% by weight of the coagulant solution. In some embodiments, the pH adjuster is used in an amount of 0.005% by weight of the coagulant solution.

In some embodiments, the coagulant solution includes the solvent. In some embodiments, the solvent includes tap water, distilled water, deionized water or combinations thereof. In some embodiments, tap water in preferably included as the solvent. In some embodiments, the solvent is used in an amount to achieve 100% by weight of the coagulant solution.

Table 3 shows an exemplary formulation of the coagulant solution.

TABLE 3
Formulation of the coagulant solution

| | Working range | Preferred range | Exemplary |
|---|---|---|---|
| Ingredients | (%) | (%) | value (%) |
| Antitack agent | 0.50 to 2.00 | 0.80 to 1.30 | 1.00 |
| Coagulating | 7.50 to 8.50 | 7.80 to 8.20 | 8.00 |
| agent | | | |
| Wetting agent | 0.01 to 0.10 | 0.03 to 0.08 | 0.05 |
| pH adjuster | 0.001 to 0.010 | 0.003 to 0.007 | 0.005 |

| | |
|---|---|
| Solvent | Used in an amount to achieve 100% by |
| | weight of the coagulant solution |

In some embodiments, the instant specification is directed to antimicrobial gloves. In some embodiments, the antimicrobial gloves include at least one layer of coating, wherein the coating is antimicrobial coating. In some embodiments, the antimicrobial coating is prepared from the above-mentioned antimicrobial coagulant solutions, or similar solutions.

In some embodiments, the antimicrobial gloves are prepared adopting the commonly known methods in the glove manufacturing industry.

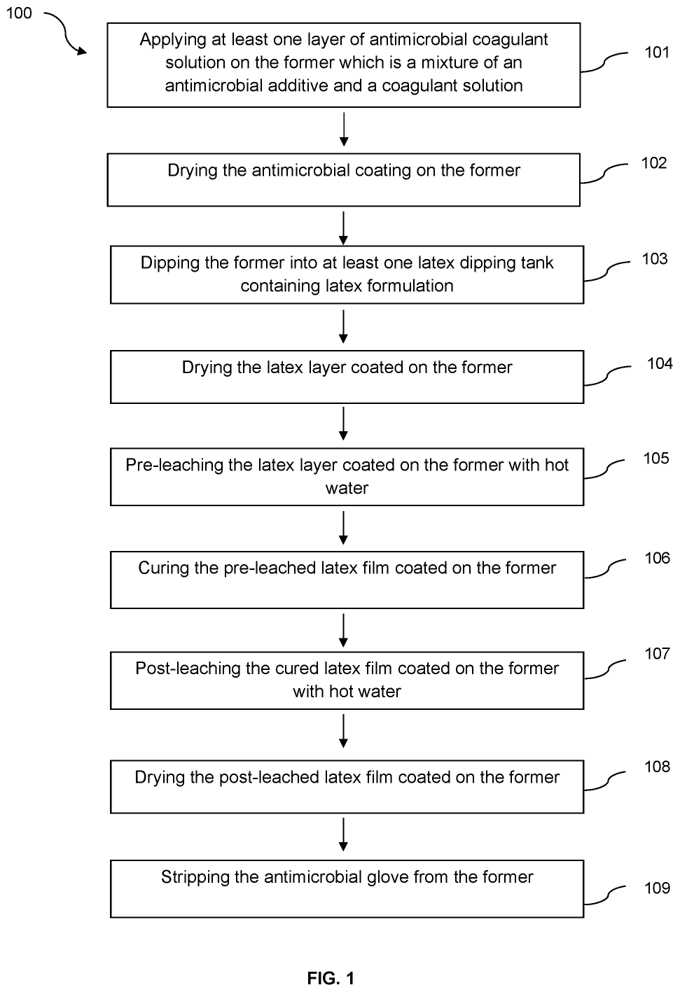

FIG. 1 shows a flow diagram of the method for producing antimicrobial gloves in accordance with some embodiments.

In some embodiments, the method to prepare the antimicrobial gloves (100) includes the steps of:

i. applying at least one layer of antimicrobial coagulant solution (such as the antimicrobial coagulant solution as described herein) on a former at a temperature ranging between 55° C. to 65° C. for a time period ranging between 10 seconds to 15 seconds to produce an antimicrobial coating on the former (101), wherein the coating may be applied by way of dipping the former into a dipping tank containing the antimicrobial coagulant solution or spraying the antimicrobial coagulant solution onto the former, wherein the coating may be carried out offline and/or online;
ii. drying the antimicrobial coating on the former obtained in step (i) at a temperature ranging between 130° C. to 140° C. for a time period ranging between 2 minutes to 3 minutes (102);
iii. dipping the former obtained in step (ii) into at least one latex dipping tank containing latex formulation at a temperature ranging between 50° C. to 65° C. for a time period ranging between 1 minute to 2 minutes to produce a latex layer coated on the former (103) wherein the latex formulation includes a base polymer, a dispersing agent, an activator, a pH adjuster, a crosslinker, an accelerator, a wetting agent, an antifoaming agent, or any combinations thereof;
iv. drying the latex layer coated on the former obtained in step (iii) at a temperature ranging between 50° C. to 70° C. for a time period ranging between 1 minute to 2 minutes (104);
v. pre-leaching the latex layer coated on the former obtained in step (iv) with hot water at a temperature ranging between 60° C. to 70° C. for a time period ranging between 1 minute to 2 minutes to leach out excess chemical residues to form pre-leached latex film (105);
vi. curing the pre-leached latex film coated on the former obtained in step (v) at a temperature ranging between 120° C. to 130° C. for a time period ranging between 12 minutes to 14 minutes to produce cured latex film (106);
vii. post-leaching the cured latex film coated on the former obtained in step (vi) with hot water at a temperature ranging between 50° C. to 60° C. for a time period ranging between 1 minute to 2 minutes to leach out excess chemical residues to obtain post-leached latex film (107);
viii. drying the post-leached latex film coated on the former obtained in step (vii) at a temperature ranging between 85° C. to 90° C. for a time period ranging between 1 minute to 2 minutes to produce antimicrobial glove (108); and
ix. stripping the antimicrobial glove obtained in step (viii) from the former (109).

The above method discusses on preparing antimicrobial glove through online process in which the antimicrobial coagulant solution is applied during online dipping process but it is not limited thereto. The antimicrobial coagulant solution can be applied through offline process in which the antimicrobial coagulant solution is applied on a glove after online process.

The antimicrobial glove as described herein can be used in a variety of applications such as but not limited to healthcare, cosmetic and/or biomedical. Additionally, teachings of the instant specification are not limited to gloves as person skilled in the art can adopt the teachings for any other elastomeric articles which exhibit similar elastomeric characteristics such as dental dam, finger cot and the like.

The antimicrobial efficacy of two exemplary antimicrobial gloves according to the instant specification (the exemplary antimicrobial glove with neem extract based coagulant coating and the exemplary antimicrobial glove with lemon extract based coagulant coating) are compared with non-antimicrobial control glove and conventional antimicrobial glove according to American Society for Testing and Materials (ASTM) D7907 standard test method. The antimicrobial efficacy of the gloves is studied against gram-positive Escherichia coli and gram-negative Staphylococcus aureus bacteria. The antimicrobial efficacy is measured based on ASTM D7907 standard test method. The results obtained are tabulated in Table 4 below.

TABLE 4
Antimicrobial efficacy of a non-antimicrobial
control glove, a conventional antimicrobial glove and
the two exemplary antimicrobial gloves
described in the instant specification

| | | Average percentage of | | | |
|---|---|---|---|---|---|
| Glove | | bacteria killing rate | | | |
| types | Bacteria | 0 min | 1 min | 3 min | 5 min |
| Non- | Staphylococcus | 12.00 | 15.76 | 18.04 | 21.99 |
| antimicrobial | aureus | | | | |
| control glove | Escherichia coli | 7.45 | 10.09 | 15.22 | 19.06 |
| Conventional | Staphylococcus | 31.25 | 40.06 | 51.17 | 62.50 |
| antimicrobial | aureus | | | | |
| glove | Escherichia coli | 24.53 | 25.47 | 27.41 | 28.30 |
| Antimicrobial | Staphylococcus | 54.00 | 65.00 | 66.00 | 67.00 |
| glove with neem | aureus | | | | |
| extract based | Escherichia coli | 10.63 | 27.66 | 34.04 | 38.29 |
| coagulant coating | | | | | |
| Antimicrobial | Staphylococcus | 42.76 | 71.99 | 96.99 | 98.87 |
| glove with lemon | aureus | | | | |
| extract based | Escherichia coli | 38.85 | 62.69 | 82.11 | 87.40 |
| coagulant coating | | | | | |

As shown in Table 4, the exemplary antimicrobial gloves described in the instant specification are able to kill both gram-positive Staphylococcus aureus and gram-negative Escherichia coli bacteria. The exemplary antimicrobial gloves according to the instant specification show higher bacteria killing rate as compared to the non-antimicrobial control glove and conventional antimicrobial glove for 1 minute, 3 minutes and 5 minutes of contact time.

As a whole, the antimicrobial gloves according to the instant specification are able to improve the antimicrobial efficacy and comply with the ASTM D7907 standard test method. The latter proves that the antimicrobial additives according to the instant specification are able to tolerate with the coagulant solution to successfully yield the antimicrobial gloves according to the instant specification.

The terminology used herein is for the purpose of describing particular example embodiments only and is not intended to be limiting. As used herein, the singular forms “a”, “an” and “the” may be intended to include the plural forms as well, unless the context clearly indicates otherwise. The terms “comprises”, “comprising”, “including”, and “having” are inclusive and therefore specify the presence of stated features, integers, steps, operations, elements, and/or components, but do not preclude the presence or addition of one or more other features, integers, steps, operations, elements, components, and/or groups therefrom.

The method, steps, processes, and operations described herein are not to be construed as necessarily requiring their performance in the particular order discussed or illustrated, unless specifically identified as an order of performance. It is also to be understood that additional or alternative steps may be employed. The use of the expression “at least” or “at least one” suggests the use of one or more elements, as the use may be in one of the embodiments to achieve one or more of the desired objects or result.

What is claimed is:

1. An antimicrobial coagulant solution, comprising
an antimicrobial additive; and
a coagulant solution,
wherein the antimicrobial additive comprises at least one selected from the group consisting of a neem extract solution and a lemon extract solution, and wherein the coagulant solution comprises an antitack agent, a coagulating agent, a wetting agent, a pH adjuster, and a solvent.

2. The antimicrobial coagulant solution of claim 1, wherein the antimicrobial additive is the neem extract solution, and an amount of the neem extract solution ranges from 0.40% to 1.00% by w/v in the antimicrobial coagulant solution.

3. The antimicrobial coagulant solution of claim 1, wherein the antimicrobial additive is the lemon extract solution, and an amount of the lemon extract solution ranges from 1.00% to 1.80% by w/v in the antimicrobial coagulant solution.

4. The antimicrobial coagulant solution of claim 1, wherein the antimicrobial additive is the neem extract solution, and wherein the neem extract solution comprises a neem extract powder and a solvent.

5. The antimicrobial coagulant solution of claim 4, wherein an amount of the neem extract powder ranges from 20.0% to 50.0% by w/v in the neem extract solution.

6. The antimicrobial coagulant solution of claim 4, wherein an amount of the solvent ranges from 50.0% to 80.0% by v/v in the neem extract solution.

7. The antimicrobial coagulant solution of claim 1, wherein the antimicrobial additive is the lemon extract solution, and wherein the lemon extract solution comprises a lemon extract powder and a solvent.

8. The antimicrobial coagulant solution of claim 7, wherein an amount of the lemon extract powder ranges from 1.0% to 20.0% by w/v in the lemon extract solution.

9. The antimicrobial coagulant solution of claim 7, wherein an amount of the solvent ranges from 80.0% to 99.0% by v/v in the lemon extract solution.

10. The antimicrobial coagulant solution of claim 1, wherein the antitack agent comprises at least one selected from the group consisting of potassium stearate and calcium stearate.

11. The antimicrobial coagulant solution of claim 1, wherein an amount of the antitack agent ranges from 0.50% to 2.00% by weight of the coagulant solution.

12. The antimicrobial coagulant solution of claim 1, wherein the coagulating agent comprises at least one selected from the group consisting of calcium nitrate and calcium chloride.

13. The antimicrobial coagulant solution of claim 1, wherein an amount of the coagulating agent ranges from 7.50% to 8.50% by weight of the coagulant solution.

14. The antimicrobial coagulant solution of claim 1, wherein the wetting agent comprises at least one selected from the group consisting of isotridecanol ethoxylates, and dihexylsulfosuccinate.

15. The antimicrobial coagulant solution of claim 1, wherein an amount of the wetting agent ranges from 0.01% to 0.10% by weight of the coagulant solution.

16. The antimicrobial coagulant solution of claim 1, wherein the pH adjuster comprises at least one selected from the group consisting of ammonia, potassium hydroxide, sodium hydroxide, magnesium hydroxide, ammonium bicarbonate, and ammonium chloride.

17. The antimicrobial coagulant solution of claim 1, wherein an amount of the pH adjuster ranges from 0.001% to 0.010% by weight of the coagulant solution.

18. The antimicrobial coagulant solution of claim 1, wherein the solvent comprises at least one selected from the group consisting of tap water, distilled water, and deionized water.

19. The antimicrobial coagulant solution of claim 1, wherein the solvent is used in an amount to achieve 100% by weight of the coagulant solution.

20. An antimicrobial elastomeric article comprising at least one layer of coating, wherein the coating is an antimicrobial coating, and wherein the antimicrobial coating is prepared from the antimicrobial coagulant solution of claim 1.

21. The antimicrobial elastomeric article of claim 20, wherein the antimicrobial elastomeric article is an antimicrobial glove.
